(19) United States
(12) Patent Application Publication
(10) Pub. No.: US 2007/0249952 A1
(43) Pub. Date: Oct. 25, 2007

(54) Systems and methods for sleep monitoring

(76) Inventors: Benjamin Rubin, Providence, RI (US); Paolo DePetrillo, Providence, RI (US); Sheldon Provost, Fremont, CA (US); Daniel Rothman, Providence, RI (US); Jason Donahue, Providence, RI (US); Julie Penner, Boulder, CO (US)

Correspondence Address: FISH & NEAVE IP GROUP;ROPES & GRAY LLP, ONE INTERNATIONAL PLACE, BOSTON, MA, 02110-2624, US

(21) Appl. No.: 11/586,196

(22) Filed: Oct. 24, 2006

Related U.S. Application Data
(63) Continuation-in-part of application No. 11/069,934, filed on Feb. 28, 2005.
(63) Continuation-in-part of application No. 11/499,407, filed on Aug. 4, 2006, now abandoned.
(60) Provisional application No. 60/729,868, filed on Oct. 24, 2005.
(60) Provisional application No. 60/729,871, filed on Oct. 24, 2005.
(60) Provisional application No. 60/729,873, filed on Oct. 24, 2005.
(60) Provisional application No. 60/729,874, filed on Oct. 24, 2005.
(60) Provisional application No. 60/729,875, filed on Oct. 24, 2005.
(60) Provisional application No. 60/729,876, filed on Oct. 24, 2005.
(60) Provisional application No. 60/548,228, filed on Feb. 27, 2004.
(60) Provisional application No. 60/705,391, filed on Aug. 4, 2005.

(51) Int. Cl. A61B 5/00

(52) U.S. Cl. 600/544

(57) ABSTRACT

Systems and methods for monitoring EEG signals include dry electrodes that can be used in sleep monitoring systems. In one aspect, a system for sleep stage monitoring and measurement includes a first dry electrode for detecting EEG signals of a user, a housing, and a sleep stage processor disposed within the housing. The first dry electrode is positioned at or near a head of the user. The sleep stage processor processes the EEG signals to determine a sleep stage of the user.

### RELATED APPLICATIONS

This application claims the benefit of and priority to U.S. provisional application No. 60/729,868, filed Oct. 24, 2005 and entitled “Electrode Placements,” U.S. provisional application No. 60/729,869, filed Oct. 24, 2005 and entitled “Real Time Sleep Stage Indicator,” U.S. provisional application No. 60/729,871, filed Oct. 24, 2005 and entitled “Circadian Modification,” U.S. provisional application No. 60/729,873, filed Oct. 24, 2005 and entitled “Multiple Users Sleep Stage Wakeup,” U.S. provisional application No. 60/729,874, filed Oct. 24, 2005 and entitled “Sleep Information and Sleep Hygiene Aid,” U.S. provisional application No. 60/729,875, filed Oct. 24, 2005 and entitled “Sleep Systems,” and U.S. provisional application No. 60/729,876, filed Oct. 24, 2005 and entitled “Bio-Sensitive Snooze Feature.” The contents of all these provisional applications are incorporated herein by reference.

This application is a continuation-in-part of U.S. patent application Ser. No. 11/069,934, filed Feb. 28, 2005 and entitled “Device for and Method of Predicting a User's Sleep State”, which claims the benefit of and priority to U.S. provisional application No. 60/548,228, filed Feb. 27, 2004 and entitled “Sleep Phase Monitor and User Responsive Awakening Device and Methods for Using Same.”

This application is also a continuation-in-part of U.S. patent application Ser. No. 11/499,407, filed Aug. 4, 2006 and entitled “Systems and Methods for Sleep Monitoring”, which claims the benefit of and priority to U.S. provisional application No. 60/705,391, filed Aug. 4, 2005 and entitled “Flexible Electrode System.” The entire teachings of the above referenced specifications are incorporated by reference herein.

### FIELD OF THE INVENTION

The invention relates to a sleep monitoring and wake-up system based on sleep stages.

### BACKGROUND

Sleep is the body's way of rejuvenating itself and is critical for learning and memory of both physical and mentally demanding tasks. Sleep is not an all-or-none phenomenon, however. Using electroencephalogram (EEG) analysis, sleep researchers have identified several distinct phases of sleep ranging from light dozing to deep sleep. These phases are marked by differing brain wave speeds and reflect different mental processes that are occurring while we sleep. For example, we dream during rapid eye movement (REM) sleep, which is characterized by rapid eye movements.

Currently, people are sleeping far less than the suggested optimal amount of an average of 8.4 hours per night due to a variety of factors, such as increased work hours, second or third jobs, longer commutes, increased media options, such as satellite television or internet websites, and family commitments. If people were getting enough sleep in their daily lives there would be little use for alarm clocks, as we would awaken naturally once the body had received enough sleep. However, since people are cutting into their optimal levels of sleep, alarm clocks are necessary to prematurely awaken sleepers.

Sleep Inertia is a phenomenon resulting from waking up without having had sufficient sleep and is roughly caused by the persistence of the physical stages of sleep into a waking state. For curtailed sleep, one of the most critical factors in determining the duration of Sleep Inertia is the sleep stage immediately preceding awakening. Abrupt awakening during deep sleep (e.g., Slow Wave Sleep or stage 3 or 4 sleep) produces greater Sleep Inertia than awakening during REM sleep or during light sleep (e.g., stage 1 or 2 sleep).

In addition, sleep debt caused by prior sleep deprivation prolongs the effects of Sleep Inertia. There is no direct evidence that Sleep Inertia is affected by a circadian rhythm. Circadian rhythm in this context means the natural twenty-four hour cycle that the human body exhibits with or without the presence of external stimuli, such as light. It seems that Sleep Inertia is more intense when awakening occurs near the core body temperature circadian trough than near its circadian peak.

A more controversial issue concerns the time course of Sleep Inertia. In a fully rested person awakened during the wrong stage of sleep, duration of Sleep Inertia may rarely exceed 30 minutes. However, because the average working person is carrying a large sleep debt, realistically, the duration of Sleep Inertia may exceed 3.5 hours, depending on the sleep state immediately preceding awakening. A more conservative middle ground estimates the duration of Sleep Inertia to be between one and two hours.

Previous attempts to remediate this problem include the “Zen alarm clock” and several “artificial dawn” clocks. The principle underlying both of these devices is first to elevate the sleeper to light sleep and then to awaken the sleeper. The Zen alarm clock uses a gradually series of bells, while the artificial dawn clock's stimulus is light of gradually increasing intensity.

Other wake-up devices include alarm clocks that attempt to detect the user's sleep state through non-EEG methods or uncomfortable EEG methods to awaken the user at the most optimal point in the sleep cycle. Many of these devices either do not accurately or scientifically gauge the user's sleep state or are not comfortable or easy to use.

### SUMMARY

The invention relates to a sleep monitoring and wake-up device with a dry electrode contact band that provides comfort, ease of use, and convenience. In one aspect of the invention, a system for sleep stage monitoring and measurement includes a first dry electrode for detecting EEG signals of a user that is positioned at or near the user's head. A sleep stage processor disposed within a housing processes the EEG signals to determine a sleep stage of the user. In one embodiment, the first dry electrode includes a conductive fabric disposed in contact with skin of the user, where the conductive fabric includes at least one of silver, copper, gold, and stainless steel. A surface of the first dry electrode in contact with skin of the user can have a surface area between 0.25 square inches and 2 square inches. A portion of the first dry electrode in contact with skin of the user can be flexible. In another embodiment, the system includes a second dry electrode for detecting the EEG signals of a user that is positioned at or near the user's head and between 0.25 inches and 1.5 inches from the first dry electrode. At least one of the first dry electrode and the second dry electrode can serve as an electrical ground.

In another embodiment, the system includes a wake-up device disposed within the housing for determining a wake-up time for the user based at least partially on the sleep stage of the user. The wake-up device can select the wake-up time according to a wake-up condition received from the user and can wake the user when the sleep stage of the user is transitioning between REM sleep and non-REM sleep. The wake-up device can include an alarm for waking the user at the wake-up time and can modify the alarm based at least partially on the sleep stage of the user in response to the alarm. The alarm can emit at least one of sound, light, vibration, and a scent. In another embodiment, the system has a second dry electrode for detecting second EEG signals of a second user. The sleep stage processor processes the second EEG signals to determine a second sleep stage of the second user. The wake-up device determines the wake-up time based at least partially on the second sleep stage of the second user.

In another embodiment, the system includes a headband adapted to encircle the user's head, attached to the first dry electrode, and for positioning the first dry electrode on the user. The headband can include a washable, stretchable fabric and the circumference formed by the headband can be adjusted to fit the user's head. At least a portion of a surface of the headband in contact with the user's head can include a non-slip material. In another embodiment, a deformable padding is disposed between the headband and the first dry electrode to press the first dry electrode into contact with the head of the user. The housing can be disposed on or near the headband.

In another embodiment, the system includes a support structure attached to the first dry electrode and for positioning the first dry electrode on the user. An electrode processor and a transmitter are disposed on or near the support structure. The electrode processor is capable of receiving the EEG signals from the first dry electrode and processing the EEG signals. The transmitter is capable of receiving the EEG signals from the electrode processor and transmitting the EEG signals. A receiver disposed within the housing is capable of receiving the EEG signals from the transmitter and transmitting the EEG signals to the sleep stage processor. The transmitter can wirelessly transmit and the receiver wirelessly receive the EEG signals. The electrode processor can amplify the EEG signals. In another embodiment, a battery charger disposed within the housing charges the electrode processor and the transmitter. In another embodiment, a support structure housing contains the electrode processor and the transmitter, and a conductive fastener attaches the support structure housing to the support structure and provides electrical communication between the first dry electrode and the electrode processor. The support structure can include at least one of a headband adapted to encircle a user's head, an eye-mask that covers the user's eyes with opaque material, eyeglasses, and an adhesive capable of adhering to the user's skin. In another embodiment, a second dry electrode detects the EEG signals of the user, is positioned at or near the user's head, and is in communication with the electrode processor. The electrode processor can generate a difference between an output of the first dry electrode and an output of the second dry electrode.

In another embodiment, the system includes a memory for storing at least one of the EEG signals and a history of sleep stages of the user. In another embodiment, the system includes a display seated on the housing for depicting information based at least partially on the sleep stage. The display can depict at least one of the EEG signals, an indicator denoting the sleep stage of the user, a hypnogram corresponding to a history of sleep stages of the user, a sleep debt number representing current sleep debt of the user, a sleep quality index representing sleep quality of the user over a period of time, a total sleep number representing a total amount of sleep over a period of time, a continuity number representing a number of arousals during a period of time, a fall asleep number representing an amount of time between placing the dry electrode on the user and when the user falls asleep, a sleep stage percentage representing a percentage of time spent by the user in a particular sleep stage, and a sleep depth number representing a depth of sleep of the user over a period of time.

The sleep stage can be at least one of light sleep, deep sleep, awake, and REM sleep.

In another embodiment, the first dry electrode detects at least one of a level of muscle tone of the user, an EOG signal, and a galvanic skin response. The sleep stage processor determines the sleep stage based at least partially on at least one of the level of muscle tone, the EOG signal, and the galvanic skin response.

In another embodiment, the system includes a sensory stimulator for providing sensory stimulation to the user to assist the user to fall asleep. The sensory stimulator can modify the sensory stimulation based at least partially on the sleep stage of the user in response to the sensory stimulation.

In another embodiment, the sleep stage processor applies a neural network when processing the EEG signals to determine the sleep stage of the user.

In another embodiment, the first dry electrode includes at least one of a capacitive electrode, a metal disk, a micromachined spike, a conductive rubber, a conductive foam, and a contactless electrode capable of detecting the EEG signals of the user without being in contact with the user.

### BRIEF DESCRIPTION OF THE DRAWINGS

The foregoing and other objects and advantages of the invention will be appreciated more fully from the following further description thereof, with reference to the accompanying drawings wherein:

FIG. 1 depicts an exemplary sleep monitoring system according to one aspect of the invention.

FIGS. 2A and 2B depict a front view and a back view, respectively, of an exemplary sensor according to one aspect of the invention.

FIG. 3 depicts a front view of an exemplary sensor similar to the sensor described with respect to FIGS. 2A and 2B.

FIGS. 4A and 4B depict a front view and a back view, respectively, of an exemplary base station according to one aspect of the invention.

FIGS. 5A and 5B depict an oblique view and a side view, respectively, of an exemplary base station similar to the base station depicted in FIGS. 4A and 4B.

FIG. 6 depicts an exemplary block diagram representing a sleep monitoring system according to one aspect of the invention.

FIG. 7 depicts a method for determining the sleep stage and a wake-up time according to one aspect of the invention that may be employed by a sleep stage processor and wake-up device.

### DESCRIPTION OF ILLUSTRATED EMBODIMENTS

To provide an overall understanding of the invention, certain illustrative embodiments will now be described. Embodiments of systems for sleep monitoring and/or wake-up devices that may monitor other physiological characteristics accessible via various locations of the user's body may also be considered within the scope of this invention. Sensors for sleep monitoring may detect electrocardiogram (ECG) signals, electromyogram (EMG) signals, breathing function or respiratory effort, and movement using wristbands, armbands, adhesive-type sensors, and/or contactless-type sensors. Systems for determining sleep stage and/or waking up the user may be portable, incorporated into other medical or consumer products, and/or unitary with any sensor systems used for monitoring characteristics of the user.

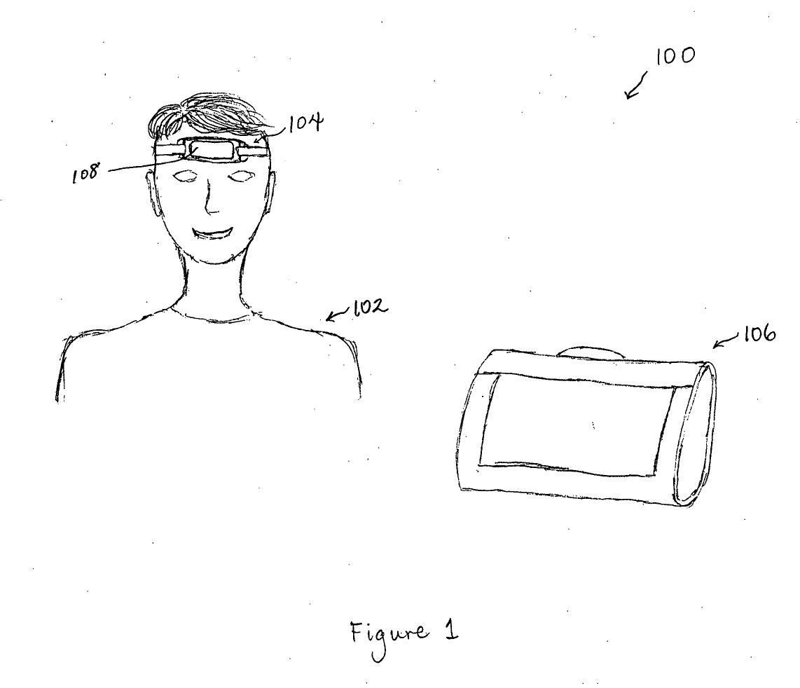

FIG. 1 depicts an exemplary sleep monitoring system 100. While sleeping, a user 102 wears a sensor 104 for detecting a biological characteristic from which the user's sleep stage can be determined. In particular, the sensor 104 includes a dry electrode placed on the user's head for detecting electroencephalogram (EEG) signals, an electrode processor for processing the EEG signals, and a wireless transmitter for transmitting an output signal of the electrode processor to a separately-housed base station 106 which serves as a bio-sensitive alarm clock.

The use of a dry electrode, which does not require any gel, paste, or preparation, and a wireless transmitter, which removes the need for a wire connecting the sensor 104 to the base station 106, enhance the user's comfort and convenience. Alternatively, the sensor 104 can use a “wet” EEG electrode to detect EEG signals and/or a wire to connect to the base station 106. The sensor 104 can also detect the level of muscle tone of the user, an electrooculogram (EOG) signal, and/or the user's galvanic skin response, which each can help determine the user's sleep stage.

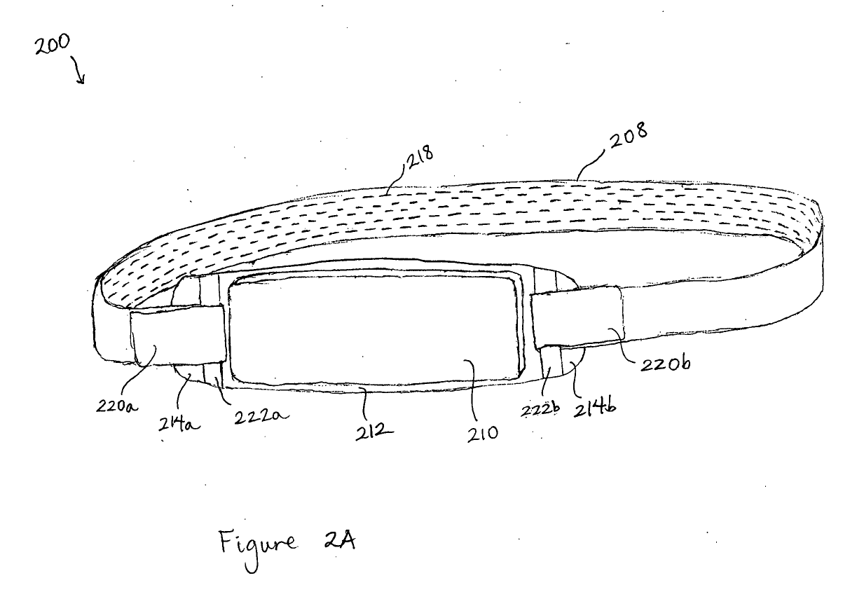

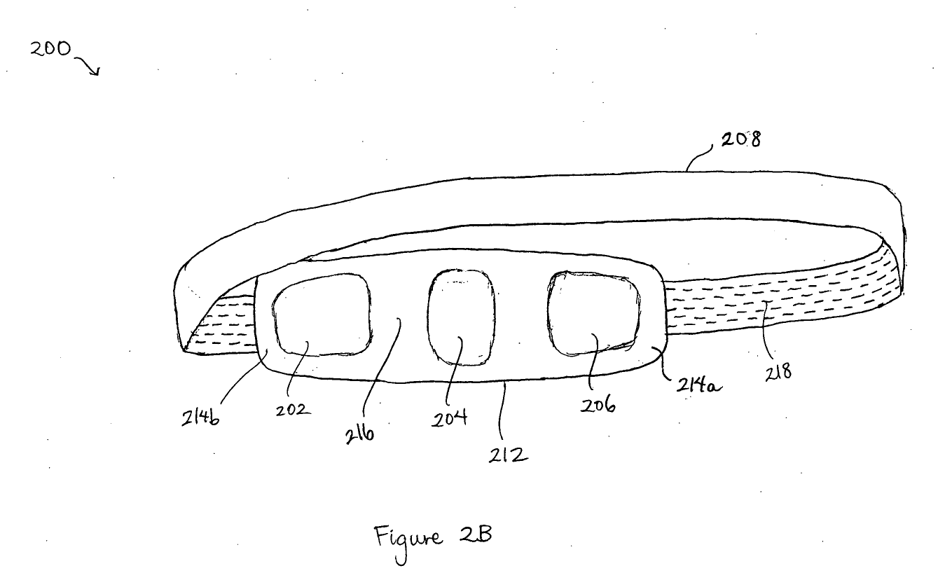

The base station 106 includes a sleep stage processor that processes the received signal to determine the user's sleep stage, a wake-up device that can wake the user based on the user's sleep stage, and a user interface for displaying sleep information to the user and for the user to indicate sleep preferences including wake-up options. Alternatively, components of the base station 106 may be incorporated into a housing 108 of the sensor 104 to create a unitary, wearable sleep monitoring system. The sensor 104 and the base station 106 are described in further detail below with respect to FIGS. 2A-5B.

The wake-up device wakes the user according to a wake-up algorithm that can depend on the user's sleep stage and sleep stage history. Generally, a user can be characterized as being awake, asleep and experiencing REM sleep, or asleep and experiencing non-REM sleep. Non-REM sleep is typically characterized as being stage 1, 2, 3, or 4, where a higher number represents deeper sleep. Stages 1 and 2 can be considered light sleep and stages 3 and 4 can be considered deep sleep. In one embodiment, the sleep processor determines that the user's sleep stage is one of light sleep, deep sleep, being awake, or REM sleep based on signals received from the sensor 104. The wake-up device generally wakes the user when the user's sleep stage is transitioning between non-REM and REM sleep, which includes transitions from non-REM to REM sleep and transitions from REM sleep to non-REM sleep, and within a window of time that includes a wake-up time set by the user. The user may also choose the length of the window of time, a preferred stage in which to be awoken, and any other constraints on how the wake-up device determines when to wake the user. For example, during a wake-up window of time, the user's sleep stage may currently be light sleep, but heading towards deep sleep, at which point the wake-up device will wake the user to avoid the user entering deep sleep. In another example, if during a wake-up window the user exits deep sleep after having been in deep sleep for a significant length of time, the wake-up device will avoid waking the user. In another example, a user may prefer to be woken from REM sleep to better recall dreams, as typically REM sleep is characterized by vivid dreaming.

FIGS. 2A and 2B depict a front view and a back view, respectively, of an exemplary sensor 200 according to one aspect of the invention. The sensor 200 includes dry electrodes 202, 204, and 206 capable of detecting EEG signals and arranged on a dry electrode backing 212. A headband 208 is attached to opposite ends 214a and 214b of the electrode backing 212 and can encircle the user's head to press the dry electrodes 202, 204, and 206 against the user's forehead, as shown in FIG. 1. A support structure housing 210, also attached to the dry electrode backing 212, contains an electrode processor for processing the EEG signals detected by the dry electrodes 202, 204, and 206 and a wireless transmitter. The dry electrodes 202, 204, and 206 are made of a conductive fabric, such as a silverized fabric. Other metals may also be used to render fabric conductive, such as copper, stainless steel, gold or a blend of copper and silver. Other dry electrodes, such as capacitive electrodes, metal disk electrodes, conductive foam, conductive rubber, and micromachined spikes, may also be used. Exemplary metal disks used in electrodes may be made of stainless steel, copper, or other metals. Exemplary foam may be silverized or otherwise made conductive, and similar to conductive fabric has the advantage of being soft and pliable. Exemplary dry rubber electrodes comprise a flexible or inflexible rubber impregnated with a conductive material such as metal or carbon nanotubes. Micromachined spikes may be made of silicon, metal, or organic materials and have the advantage of being able to penetrate the layer of skin that impedes signal transmission. Exemplary dry electrodes that are capacitive as opposed to ohmic, exemplary conductive foam, and exemplary metal disk electrodes are described in “Dry and Capacitive Electrodes for Long-Term ECG-Monitoring” by Anna Karilainen, Stefan Hansen, and Jörg Muiller, SAFE2005, 8th Annual Workshop on Semiconductor Advances for Future Electronics, 17-18 Nov. 2005, Veldhoven, The Netherlands, p.155-161. Exemplary capacitive electrodes that do not require contact with the user's skin are described in “Remote detection of human electroencephalograms using ultrahigh input impedance electric potential sensors,” by C. J. Harland, T. D. Clark, and R. J. Prance, Applied Physics Letters, Vol. 81, No. 17, Oct. 21, 2002, p. 3284-3286. Exemplary micromachined spikes are described in “Characterization of Micromachined Spiked Biopotential Electrodes” by Patrick Griss, Heli K. Tolvanen-Laakso, Pekka Meriläinen, and Göran Stemme, IEEE Transactions on Biomedical Engineering, Vol. 49, No. 6, June 2002, p. 597-604. The above references are hereby incorporated by reference herein.

Dry electrodes enhance user convenience and comfort by removing the need for gels, pastes, or other preparation. Conductive fabric dry electrodes in particular are soft and pliable, which further enhances user comfort. In general, dry electrodes having surfaces in contact with the user that are flexible may be more comfortable for the user to wear while sleeping. A deformable padding, such as a deformable foam, may be disposed behind each dry electrode to help press the dry electrode against the user's skin. In one embodiment, each dry electrode 202, 204, and 206 can have a surface area in contact with the user's skin approximately between 0.25 square inches and 2 square inches. The dry electrode backing 212 arranges the dry electrodes 202, 204, and 206 at a distance approximately between 0.25 inches and 1.5 inches from each other. A back surface 216 of the dry electrode backing 212 can be made of a felt-like or otherwise soft material to enhance user comfort. A portion of the back surface 216 may also include a non-slip material to impede movement or slipping of the dry electrodes 202, 204, and 206 relative to the user. In one embodiment, the center dry electrode 204 represents electrical ground and the electrode processor generates a difference between the signals detected by the side electrodes 202 and 206. Any number of dry electrodes may also instead be used. A metal sheet layer in communication with a ground electrode, such as the center dry electrode 204, may be disposed beneath the back surface 216 to electrically shield the dry electrodes.

The headband 208 can be made of elastic, or other flexible fabric or materials that are also stretchable, to allow the headband 208 to fit snugly around the user's head. The circumference formed by the headband 208 may also be adjusted to ensure a comfortable and useful fit. In particular, opposing ends 220a and 220b of the headband 208 may thread through backing loops 222a and 222b, respectively, and double back to fasten to the headband 208 with hook-and-loop fasteners such as Velcro®, or any other reusable fastener. The length of the ends 220a and 220b of the headband 208 that are doubled back may be varied to vary the circumference formed by the headband 208. The headband 208 may also be removed entirely from the electrode backing 212 to be washed or replaced. The headband 208 may include non-slip material, such as rubber 218, that is less likely to allow the headband, and thus the dry electrodes as well, to move or slip when worn by the user. Other support structures may also be used to position and secure the dry electrodes 202, 204, and 206 other than the headband 208, such as an eye-mask that covers the user's eyes with opaque material to block light from reaching them, eyeglasses, and an adhesive to position and secure the electrode backing 212 to the user. Further description and other electrode and support structure embodiments are described in U.S. patent application Ser. No. 11/499,407, filed Aug. 4, 2006 and entitled “Systems and Methods for Sleep Monitoring” and in U.S. Provisional Application No. 60/729,868, filed Oct. 24, 2005 and entitled “Electrode Placements.”

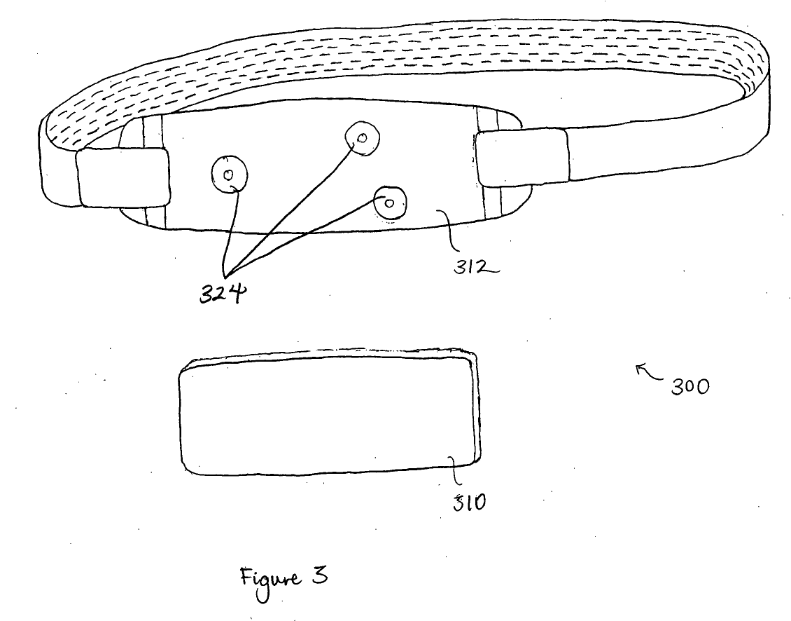

FIG. 3 depicts a front view of an exemplary sensor 300 similar to the sensor 200 described with respect to FIGS. 2A and 2B. The sensor 300 has a support structure housing 310 that is attached to a dry electrode backing 312 by metal snaps 324, which also serve to conduct signals from dry electrodes (not shown) arranged on the opposing surface of the dry electrode backing 312 to an electrode processor contained within the support structure housing 310.

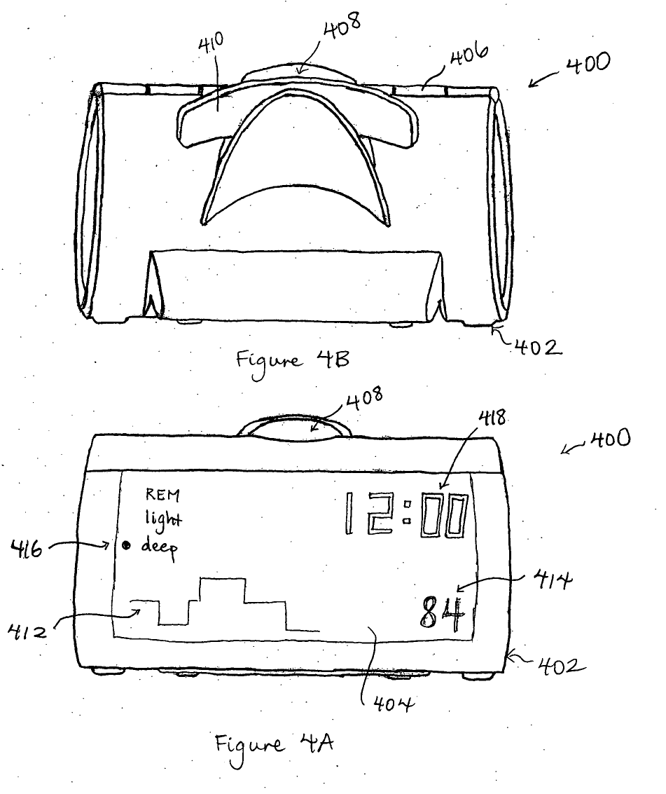

FIGS. 4A and 4B depict a front view and a back view, respectively, of an exemplary base station 400 according to one aspect of the invention. The base station 400 has a housing 402 that includes a display 404, an array of buttons 406, and a charging area 408 configured to hold and charge a sensor 410, similar to the sensors 104, 200, and 300 described above. The housing 402 contains a receiver for receiving signals from the sensor 410, a sleep stage processor for determining the user's sleep stage, and a wake-up device for determining when to wake the user, based at least partially on the user's sleep stage.

The housing 402 can be made of plastic, or any other suitably lightweight, rigid material, and have a height, width, or depth approximately between 0.25 inches and 12 inches. In one implementation, the housing 402 is about 4 inches high, 6 inches wide, and 2 inches deep. The display 404 of the housing 402 can be any suitable display screen such as an LCD, MEMS-based, or LED display. The display 404 can show, in addition to information usually shown by an alarm clock, such as the time 418 and if any alarms are set, information related to the user's sleep such as a hypnogram 412 representing a sleep history, sleep debt, total sleep, time to fall asleep, number of arousals, percentage of time spent in a particular sleep stage, sleep intensity or depth, the current sleep stage 416, and a sleep quality index 414. The hypnogram 412 can depict the user's sleep history throughout one night either by a wavy line representing sleep depth versus time or by a step function representing sleep stage versus time. The latter representation may be generated by averaging sleep depth over an interval of time, for consecutive intervals of time, to determine one sleep stage per interval, which is then depicted as a step function graph whose different heights represent different sleep stages. Sleep debt represents a current total amount of sleep over one or multiple nights that the user is below a desired norm, such as 8 hours of sleep per night or some other goal for average amount of sleep per night entered by the user. Total sleep represents the total amount of sleep achieved by the user during one sleep session, from the moment the user dons the sensor 410 to the current time, excluding any time spent not sleeping. Time to fall asleep represents the amount of time from the moment the user dons the sensor 410 till the user falls asleep for the most recent sleep session. The number of arousals represents the number of times the user woke up during a sleep session for a significant period, for example for more than one minute. Sleep intensity or depth represents how deep the sleep was during a sleep session, and can be represented as a percentage of time spent in deep sleep. The current sleep stage 416 allows others to see whether or not the user is sleeping or in a stage at which he/she can be awoken. The sleep quality index 414 is a number representing the quality of a sleep session, such as a number between 1 and 100, that may be based on information related to the user's sleep, such as any of the above information.

The sleep stage processor may also be configured to accept signals from sensors being worn by different users to determine a sleep stage for each user. The wake-up device may then adjust the wake-up algorithm to wake up both users at a point in time within a wake-up window that is desirable for both users based on their respective sleep stages. For example, if one user is transitioning between REM and non-REM sleep, then the wake-up device can decide to wake them if the other user is in light sleep or to not wake them if the other use is in deep sleep.

The base station 400 can be capable of emitting sensory stimulation, such as sound, light, tactile sensations such as vibration, or scents, to adjust the user's sleep state. The wake-up device can have an alarm, similar to those available for alarm clocks, that wakes the user according to wake-up algorithms as described in reference to FIGS. 1 and 4. The volume and/or intensity of the alarm may be modified in response to the user's detected sleep stage. Sensory stimulation may also be used to encourage the user to fall asleep, and may also be modified in response to the user's detected sleep stage. For example, soothing sounds may be played that gradually diminish in volume when the user's sleep stage indicates that they have fallen asleep. In another example, the intensity of a light may dim at a rate approximately proportional to the user's sleep stage. In another example, music can entrain the user's brain to move into a particular state based on the music's frequency or other characteristics.

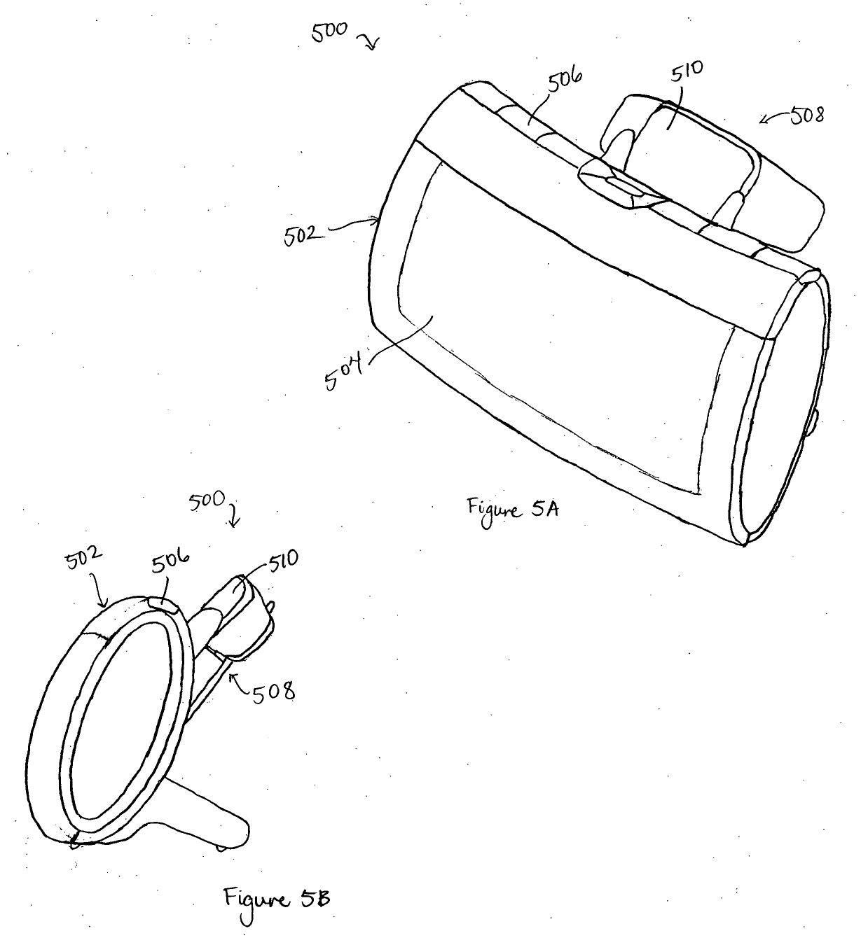

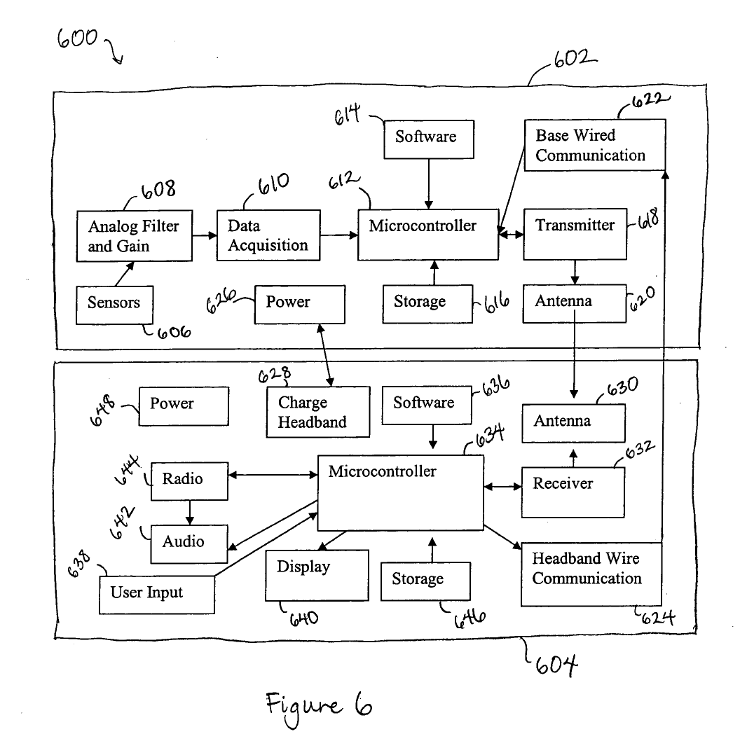

FIGS. 5A and 5B depict an oblique view and a side view, respectively, of an exemplary base station 500 similar to the base station 400 depicted in FIGS. 4A and 4B. The base station 500 has a housing 502 that includes a display 504, an array of buttons 506, and a charging area 508 configured to hold and charge a sensor 510. The housing 502 contains a receiver for receiving signals from the sensor 510, a sleep stage processor for determining the user's sleep stage, and a wake-up device for determining when to wake the user, based at least partially on the user's sleep stage.

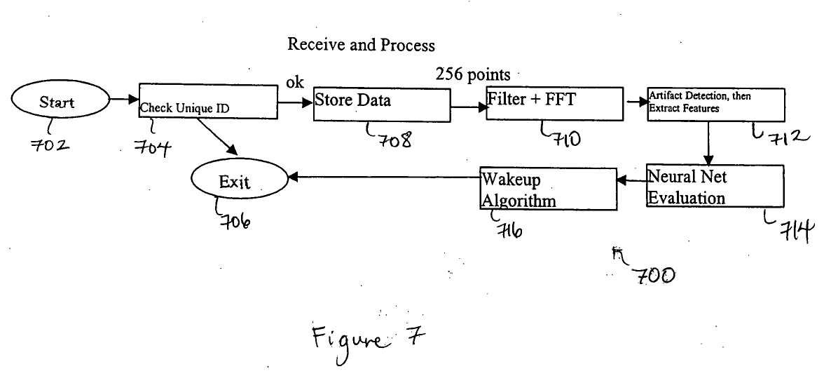

FIG. 6 depicts an exemplary block diagram representing a sleep monitoring system 600 according to one aspect of the invention. A sensor system 602 can be disposed near or within a support structure housing similar to the support structure housings 210 and 310 depicted in FIGS. 2A-B and 3, respectively. A base station system 604 can be disposed on or within a housing similar to the housings 402 and 502 depicted in FIGS. 4A-B and 5A-B, respectively.

The sensor system 602 includes sensors 606, such as dry electrodes capable of detecting EEG signals, which transmit signals detected from the user to an electrode processor. The electrode processor includes analog filtering and gain circuitry 608 which can filter and amplify signals received from the sensors 606. In one embodiment, the circuitry 608 filters through frequencies approximately between 2 Hz and 50 Hz, to filter out non-EEG signals, and, using a differential amplifier, amplifies by approximately 5000 times a signal representing the difference between signals measured from two electrodes. A microcontroller 612, controlled by software 614, can convert the processed analog signal to a digital signal that can be stored in a memory or storage buffer 616 while awaiting transmission via a transmitter 618 in communication with an antenna 620 for wirelessly transmitting signals.

These signals are wirelessly received by the base station system 604 by an antenna 630 in communication with a receiver 632 which transmits the received signals to a microcontroller 634 controlled by software 636 to determine a wake-up time and/or whether to emit and/or modify sensory stimulation. The microcontroller can receive user input 638 that can be entered using buttons, such as buttons 406 and 506 depicted in FIGS. 4A-B and 5A-B, respectively, and help determine a wake-up condition or time, sleep-related information to depict on a display 640, and what sensory stimulation to provide via a light or audio device 642, which can play sound from a radio 644. The microcontroller 634 can store on a memory or storage buffer 646 signals received by the receiver 632, preferences indicated by the user via user input 638, and other sleep-related information such as a sleep history. The components of the base station system 604 can be powered by a power supply 648.

The microcontroller 612 of the sensor system 602 can measure the impedance between two electrodes of the sensors 606 by transmitting a signal through one electrode that is measured by the other. This impedance check can detect that there is no connection between the two electrodes, meaning the sensors 606 are not in contact with the user, which allows the sensor system 602 to turn off to conserve power. The measured impedance may also be used to adjust how the signals are processed and how the sleep stage is determined. A low impedance may suggest that the signals detected by the electrodes are of a good quality, and conversely a high impedance may suggest that the signals are of a poor quality. Different impedances may also correlate with specific types of noise.

The sensor system 602 includes a base wired communication component 622 capable of interfacing with a headband wire communication component 624 in the base station system 604 to communicate when to recharge a power supply 626 of the sensor system 602 using a battery charger 628 in the base station system 604. In particular, when the user places the sensor system 602 on a charging area of a base station, such as charging areas 408 and 508 depicted in FIGS. 4 and 5, respectively, the base wired communication component 622 detects that the sensor system 602 is engaged with the base station, then wirelessly communicates this information to the base station. In response, the base station can, depending on the voltage available on the power supply 626 of the sensor system 602, either charge the power supply 626 or indicate that the power supply 626 is fully charged, e.g. via the display 626.

FIG. 7 depicts a method 700 for determining the sleep stage and a wake-up time according to one aspect of the invention that may be employed by a sleep stage processor and wake-up device. A base station system, such as the system 604 depicted in FIG. 6, receives a wireless signal at the start 702 and checks the unique ID at step 704 to verify whether or not the received signal comes from the correct sensor system, such as sensor system 602 depicted in FIG. 6. If the signal does not have the correct ID, then the method ends at step 706. If the ID is correct, then the sleep stage processor stores the received data at step 708. After a certain number of samples are received, for example 256 data points, the sleep stage processor filters and converts the data to the frequency domain, for example by applying a fast fourier transform (FFT), at step 710. At step 712, the sleep stage processor may also perform artifact detection to determine the quality of the signal, for example whether or not it adequately represents an EEG signal, and to extract features from the signal, such as delta, theta, and beta waves of an EEG signal. The extracted features may then be communicated to a neural network at step 714 which evaluates the features to determine a corresponding sleep stage. The sleep stage determined by the neural network is communicated to a wake-up algorithm of a wake-up device at step 716, which determines when to wake the user up based on the sleep stage.

The foregoing embodiments are merely examples of various configurations of components of sleep monitoring systems described and disclosed herein and are not to be understood as limiting in any way. Additional configurations can be readily deduced from the foregoing, including combinations thereof, and such configurations and continuations are included within the scope of the invention. Variations, modifications, and other implementations of what is described may be employed without departing from the spirit and the scope of the invention. More specifically, any of the method, system and device features described above or incorporated by reference may be combined with any other suitable method, system, or device features disclosed herein or incorporated by reference, and is within the scope of the contemplated inventions.

We claim:

1. A system for sleep stage monitoring and measurement, comprising
a first dry electrode for detecting EEG signals of a user, positioned at or near a head of the user,
a housing, and
a sleep stage processor disposed within the housing for processing the EEG signals to determine a sleep stage of the user.

2. The system of claim 1, wherein the first dry electrode comprises a conductive fabric disposed in contact with skin of the user.

3. The system of claim 2, wherein the conductive fabric includes at least one of silver, copper, gold, and stainless steel.

4. The system of claim 1, wherein a surface of the first dry electrode in contact with skin of the user has a surface area between 0.25 square inches and 2 square inches.

5. The system of claim 1, wherein a portion of the first dry electrode in contact with skin of the user is flexible.

6. The system of claim 1, comprising a second dry electrode for detecting the EEG signals of a user, positioned at or near a head of the user and between 0.25 inches and 1.5 inches from the first dry electrode.

7. The system of claim 6, wherein at least one of the first dry electrode and the second dry electrode serves as an electrical ground.

8. The system of claim 1, comprising a wake-up device disposed within the housing for determining a wake-up time for the user based at least partially on the sleep stage of the user.

9. The system of claim 8, wherein the wake-up device selects the wake-up time according to a wake-up condition received from the user and to wake the user when the sleep stage of the user is transitioning between REM sleep and non-REM sleep.

10. The system of claim 8, wherein
the wake-up device comprises an alarm for waking the user at the wake-up time, and
the wake-up device modifies the alarm based at least partially on the sleep stage of the user in response to the alarm.

11. The system of claim 8, wherein the wake-up device comprises an alarm for waking the user at the wake-up time and capable of emitting at least one of sound, light, vibration, and a scent.

12. The system of claim 8, comprising a second dry electrode for detecting second EEG signals of a second user, wherein
the sleep stage processor processes the second EEG signals to determine a second sleep stage of the second user, and
the wake-up device determines the wake-up time based at least partially on the second sleep stage of the second user.

13. The system of claim 1, comprising a headband adapted to encircle a head of the user, attached to the first dry electrode, and for positioning the first dry electrode on the user.

14. The system of claim 13, wherein the headband comprises a washable, stretchable fabric.

15. The system of claim 13, wherein a circumference formed by the headband can be adjusted to fit the head of the user.

16. The system of claim 13, wherein at least a portion of a surface of the headband in contact with the head of the user comprises a non-slip material.

17. The system of claim 13, comprising a deformable padding disposed behind the first dry electrode to press the first dry electrode into contact with the head of the user.

18. The system of claim 13, wherein the housing is disposed on or near the headband.

19. The system of claim 1, comprising
a support structure attached to the first dry electrode and for positioning the first dry electrode on the user,
an electrode processor disposed on or near the support structure and capable of receiving the EEG signals from the first dry electrode and processing the EEG signals,
a transmitter disposed on or near the support structure and capable of receiving the EEG signals from the electrode processor and transmitting the EEG signals, and
a receiver disposed within the housing and capable of receiving the EEG signals from the transmitter and transmitting the EEG signals to the sleep stage processor.

20. The system of claim 19, wherein the transmitter wirelessly transmits the EEG signals and the receiver wirelessly receives the EEG signals.

21. The system of claim 19, wherein the electrode processor amplifies the EEG signals.

22. The system of claim 19, comprising a battery charger disposed within the housing for charging the electrode processor and the transmitter.

23. The system of claim 19, comprising
a support structure housing containing the electrode processor and the transmitter, and
a conductive fastener attaching the support structure housing to the support structure and providing electrical communication between the first dry electrode and the electrode processor.

24. The system of claim 19, wherein the support structure comprises at least one of a headband adapted to encircle a head of the user, an eye-mask that covers eyes of the user with opaque material, eyeglasses, and an adhesive capable of adhering to skin of the user.

25. The system of claim 19, comprising a second dry electrode for detecting the EEG signals of the user, positioned at or near the head of the user, and in communication with the electrode processor.

26. The system of claim 25, wherein the electrode processor generates a difference between an output of the first dry electrode and an output of the second dry electrode.

27. The system of claim 1, comprising a memory for storing at least one of the EEG signals and a history of sleep stages of the user.

28. The system of claim 1, comprising a display seated on the housing for depicting information based at least partially on the sleep stage.

29. The system of claim 28, wherein the display depicts at least one of the EEG signals, an indicator denoting the sleep stage of the user, a hypnogram corresponding to a history of sleep stages of the user, a sleep debt number representing current sleep debt of the user, a sleep quality index representing sleep quality of the user over a period of time, a total sleep number representing a total amount of sleep over a period of time, a continuity number representing a number of arousals during a period of time, a fall asleep number representing an amount of time between placing the dry electrode on the user and when the user falls asleep, a sleep. stage percentage representing a percentage of time spent by the user, in a particular sleep stage, and a sleep depth number representing a depth of sleep of the user over a period of time.

30. The system of claim 1, wherein the sleep stage is at least one of light sleep, deep sleep, awake, and REM sleep.

31. The system of claim 1, wherein
the first dry electrode detects at least one of a level of muscle tone of the user, an EOG signal, and a galvanic skin response,
and the sleep stage processor determines the sleep stage based at least partially on at least one of the level of muscle tone, the EOG signal, and the galvanic skin response.

32. The system of claim 1, comprising a sensory stimulator for providing sensory stimulation to the user to assist the user to fall asleep.

33. The system of claim 32, wherein the sensory stimulator modifies the sensory stimulation based at least partially on the sleep stage of the user in response to the sensory stimulation.

34. The system of claim 1, wherein the sleep stage processor applies a neural network when processing the EEG signals to determine the sleep stage of the user.

35. The system of claim 1, wherein the first dry electrode comprises at least one of a capacitive electrode, a metal disk, a micromachined spike, a conductive rubber, a conductive foam, and a contactless electrode capable of detecting the EEG signals of the user without being in contact with the user.
